(19) United States
(12) Patent Application Publication
(10) Pub. No.: US 2011/0250238 A1
(43) Pub. Date: Oct. 13, 2011

(54) SYSTEM FOR THE COLON DELIVERY OF DRUGS SUBJECT TO ENZYME DEGRADATION AND/OR POORLY ABSORBED IN THE GASTROINTESTINAL TRACT

(76) Inventors: Maria Edvige Sangalli, Pavia (IT); Gianaldo Tavella, Pavia (IT); Maria Dorly Del Curto, Corvino SanQuirico (IT); Andrea Gazzaniga, Santa Giuletta (IT); Alessandra Maroni, Luvinate (IT); Luca Palugan, Magnago (IT); Lucia Zema, Como (IT); Maddalena Spreafico, Galbiate (IT)

(21) Appl. No.: 13/054,149

(22) PCT Filed: Jul. 17, 2009
(86) PCT No.: PCT/IB2009/006266

§ 371 Date: Mar. 25, 2011

(30) Foreign Application Priority Data
Jul. 18, 2008 (IT) MI2008A001313

(51) Int. Cl. A61K 9/00; A61K 38/28; A61P 1/00; A61K 31/575; A61P 3/10; A61K 38/02; A61K 31/24

(52) U.S. Cl. 424/400; 514/1.1; 514/5.9; 514/533; 514/171; 514/182

(57) ABSTRACT

Said invention refers to a pharmaceutical form for selective colon delivery of drugs or bioactive molecules degraded and/or poorly absorbed in the gastrointestinal tract. The system comprises a core consisting of the active ingredient, and a protease inhibitor layer and/or an absorption enhancer layer, said core being separated from these layers by means of a polymer that swells and/or dissolves and/or is degraded when in contact with the biological fluids present in the gastro-intestinal tract; depending on the thickness of the polymeric layer, the release of the drug can be modulated with respect to the inhibitor and/or promoter.

### FIELD OF INTEREST

The present invention concerns a pharmaceutical dosage form for selective colon delivery of drugs or bioactive molecules degraded and/or poorly absorbed in the gastro-intestinal tract, and in particular peptides and proteins.

### BACKGROUND OF THE INVENTION

Oral formulations for drug site-directed release could be conveniently used to allow the administration of drugs that undergo degradation processes by the enzymes present in the gastro-intestinal tract. The colon offers a more favourable environment for the absorption of these drugs as the presence of digestive enzymes is limited as compared with the small intestine. Nonetheless, in order to obtain adequate pharmacological responses, especially when dealing with, proteins and peptides, formulations should also include protease inhibitors and/or absorption promoters.

Based on these premises, Katsuma et al. (Int. J. Pharm. 307, 156-162 2006) have recently described an insulin colonic release system containing camostat mesilate as a protease inhibitor and sodium glycocholate, sodium lauryl sulfate and di-sodium EDTA as absorption promoters; in an in vivo study performed on dogs, such systems elicited an enhanced hypoglycaemic effect as compared with those systems in which no adjuvants were included.

US 2004/0185107 refers to an oral pharmaceutical dosage form composed of an erodible polymeric matrix able to concurrently release the active principle along with the protease inhibitor and/or absorption promoter, claiming a number of advantages over a system from which the release of protein and promoter and/or inhibitor fails to be programmed.

On the other hand, some authors (Dorkoosh F. A. et al. J. Contr. Rel. 71, 307-318 2001) introduced the concept of the so-called “double phase time release profile” relevant to a system composed of a bioadhesive polymer capable of releasing in the small intestine a protease inhibitor and/or an absorption promoter, prior to the release of the drug. In particular, the possibility to inhibit protease activity and to modify the epithelium characteristics during the time when the polymer swells, thus prior to the peptide release, is claimed.

However, such systems are designed for release in the small intestine, which is proven to be a by far less favourable site for the absorption of proteins and/or peptides with respect to the large intestine. Furthermore, due to the complex manufacturing, such system present poor industrial scale-up prospects.

On the other band, the device described in EP 0572942 is capable of releasing drugs, including proteins, into the colon (Serratoni M. et al., Proceed. Int'l Symp. Control. Rel. Bioact. Mater. 34, 749 2007) using a technology carried out in industrial equipments on pilot scale which avoids the use of organic solvents, thus showing scalability advantages. According to EP 0572942, this system comprises:
a) a core containing an active ingredient optionally in admixture with pharmaceutically acceptable excipients;
b) an intermediate coating layer comprising a hydrophilic erodible polymer that delays the release of the active ingredient contained in the core in a pH-independent fashion;
c) an outer layer, the dissolution of which triggers the erosion process of the intermediate layer,

in which the intermediate layer is applied in such way that the weight gain with respect to the core is between 10 and 300%.

In the literature, other documents may be found with no or negligible pertinence to the field of the present invention, which are briefly described below.

WO 2004/05/602 describes a formulation provided with an outer gastroresistant coating consisting of a polymer insoluble at acidic pH values, and containing a protein drug along with a protease inhibitor. The mentioned formulations, however, are not designed for a time-dependent release, as they do not include components able to delay the release of the active ingredient and the adjuvant for a programmed time period. The formulation is neither intended for a colon delivery, nor for a time-dependent colon delivery.

US 2002/004495 describes a system designed for colon delivery comprising a drug-containing core coated with a polymer with pH-dependent solubility, soluble at acidic pH values, and with an outer saccharidic layer susceptible to be selectively degraded by the enterobacteria present in the distal intestine. The degradation of the saccharidic layer, resulting in the formation of organic acids, causes the dissolution of the underlying pH-dependent layer. The selective colon release is thus achieved by exploiting the peculiar distribution of the colonic microflora. Said document makes no reference to a time-dependent release or to the use of swellable hydrophilic polymers in order to achieve such goals. Furthermore, no reference is made to the possibility of modulating the release of an adjuvant (enzymatic inhibitor/absorption promoter) with respect to the conveyed drug.

WO 03/059330 discloses a multi-layered system meant for the therapy of cardiovascular disease. The drug contained in the core is not a protein. The molecule conveyed in a coating layer surrounding the core is intended to inhibit a proteolytic enzyme, yet such enzyme is not inhibited in the gastrointestinal tract and its inhibition is not aimed at preserving the stability of orally administered drugs. The formulations described in this document do not contain any protein drug in a core or intestinal digestive protease inhibitor conveyed in a coating layer.

US 2007/0172525 reports on a pharmaceutical formulation intended for the therapy of type 2 diabetes. The core, designed for slow release, contains a non-protein drug, particularly metformin, belonging to the biguanide category. A second active ingredient belonging to the dipeptidylpeptidase-4 (DPP4) inhibitor family, is found in an outer coating layer. Both drug categories, biguanides and DPP4-inhibitors, are oral hypoglycemics. This is another case in which the inhibition of an enzyme does not constitute a strategy for increasing the intestinal stability of orally administered drugs.

U.S. Pat. No. 4,101,650 describes a formulation intended for the therapy of gastric and duodenal ulcer consisting of mini-capsules that are designed for a prolonged gastric residence enabled by the relevant floating properties. Such mini-capsules comprise a granule of sodium bicarbonate, a film coating rapidly soluble in aqueous medium and a surrounding layer containing pepstatin. The carbon dioxide produced inside the units as a result of the contact between sodium bicarbonate and the acidic aqueous medium allows them to float on the surface of the gastric content, thus being trapped in the stomach for longer time periods as compared with conventional formulations. Pepstatin released in the gastric lumen inhibits pepsin, a protease involved in the digestion of dietary proteins, yet again not aiming at improving the stability of co-administered drugs but at yielding a specific therapeutic effect.

US 2005/026608 refers to a system for the release of DPP4-inhibitors in the therapy of type 2 diabetes. Such system comprises a placebo core, in which no drug is present, a first sealing layer, an active layer containing the active DPP4-inhibitor and an outer sealing layer, The two sealing layers are exclusively intended to protect the drag from the degradation the inventors hypothesize could be induced by the contact with tablet excipients.

WO 03/0395118 describes various extended-release compositions of nicotine or derivatives thereof. Among the possible configurations proposed for the system, the one described in Example 1 comprises, starting from the outer layer to the inner one, a first layer (layer 1) containing sodium bicarbonate meant to buffer the pH at values favourable for drug absorption in the oral cavity, a second layer (layer 2) with a sealing function (“physical barrier”), a third layer (layer 3) containing an immediate release formulation of the drug and, finally, a core also containing the drug but designed for prolonged release (layer 4). The core does not contain any active molecule for which a site-selective release in, the distal intestine would be beneficial. Furthermore, no coating is present that can delay the onset of the conveyed drug release.

EP 0 636 366 discloses a multi-layer system for prolonged release of drugs, namely opiate analgesics, that can present “overcoat” layers consisting of HPMC but applied exclusively as protective films, with thicknesses that are not suited for the time of drug release to be modified.

U.S. Pat. No. 4,579,730 describes a formulation intended for the oral co-administration of insulin and adjuvants, such, as absorption promoters and protease inhibitors. In any case, this invention does not refer to any structural element intended to delay the time of drug release or enable a preferential colonic release.

### DESCRIPTION OF THE INVENTION

It has now been found that, by separating the active ingredient-containing core from a layer containing said inhibitors and/or promoters by means of a layer of polymers that swell and/or dissolve and/or are degraded when in contact with aqueous fluids, it is possible to achieve a release of the inhibitor and/or promoter prior to the release of the active ingredient contained in the core. The time elapsed from the release of the protease inhibitor and/or absorption enhancer to the release of the active ingredient is programmable as a function of the thickness of the interposed polymeric layer; thus, the drug absorption in the colon is improved because, as the protease inhibitor and/or absorption enhancer are released prior to the drug, the proteolytic enzymes are already inhibited and intestinal epithelium is already modified when the drug is released.

The present invention therefore refers to orally administered solid pharmaceutical forms, shown schematically in FIG. 1, that comprise:
a) a core containing a drug susceptible to enzymatic degradation and/or poorly absorbed in the gastrointestinal tract;
b) an inner layer consisting of a polymer or a mixture of polymers which swell and/or dissolve and/or are degraded upon contact with the biological fluids in the gastrointestinal tract;
c) an intermediate layer consisting of a protease inhibitor and/or an absorption enhancer, optionally in admixture with a binding polymer or other pharmaceutically acceptable excipients;
d) an outer layer consisting of a polymer or a mixture of polymers which swell and/or dissolve and/or are degraded upon contact with the biological fluids in the gastrointestinal tract, which can be the same as or different from those which form layer b); and optionally
e) an, outer layer consisting of a gastro-resistant polymer

wherein the thickness of layer b) ranges from 10 to 1350 μm, preferably from 150 to 450 μm; and

the thickness of layer c) ranges from 10 to 1000 μm, preferably from 10 to 300 μm; and the thickness of layer d) ranges from 150 to 1500 μm, preferably from 200 to 1000 μm.

It has been proven that, the delay observed in the release of the active ingredient with respect to said inhibitors and/or enhancers is directly proportional to the thickness of layer b).

The active ingredient contained within the core may consist of a peptide or protein, for example insulin, vasopressin and analogues, calcitonin, enkephalins, cyclosporines, oxytocin, Follicle-Stimulating Hormone (FSH), Luteinizing Hormone (LH), superoxydodismutase, interleukin 12 (IL-12), interferons, Colony Stimulating Factor (CSF), Tumor Necrosis Factor (TNF), Gonadotropin Releasing Hormone (GnRH)-antagonists, monoclonal antibodies such as cetuximab, bevacizumab and also other non-protein drugs, for example chlorpromazine, ethinylestradiol, flurazepam and lorazepam, that undergo degradation when in contact with the gastro-intestinal enzymes. The core generally consists of a tablet of active ingredient blended with pharmaceutically acceptable excipients, as the ones described in Remington's 21^{st} Ed. or in the Handbook of Excipients 5^{th} Ed. A. H. Kibbe Editors Washington, D.C., 2000, chosen according to the properties of the active ingredient, the type of dosage form and the release characteristics that are expected from the core. The core may also consist of capsules, minitablets and pellets.

The polymers composing layers b) and d) are selected from:
1. hydrophilic swellable and/or erodible polymers, such as cellulose derivatives, e.g. methylcellulose (MC), carboxymethylcellulose (CMC), hydroxyethylcellulose (HEC), hydroxypropyl methylcellulose (HPMC), hydroxypropylcellulose (HPC);
2. natural hydrophilic polymers degraded by the intestinal microflora, such as guar gum, dextrans, pectinates, alginates, hyaluronates, lactulose, amylose, amylopectin cellobiose, fructo-oligosaccharides, chitosans, cyclodextrins and chondroitin sulfates.
3. synthetic hydrophilic polymers, such as polyvinyl alcohols, polyethylene glycols, polyvinylpolypyrrolidones, acrylic polymers, such as polycarbophil, carbopol, copolymers of methacrylic acid, e.g. Eudragit RL, Eudragit RS, Eudragit FS, Eudragit S, Eudragit L, Eudragit NE.

Furthermore, the polymeric layer may contain one or more pharmaceutically acceptable excipients, chosen, for example, among those indicated in Handbook of Excipients 5^{th} Ed. A. H. Kibbe Editors Washington, D.C., 2000, such as magnesium stearate, glyceryl behenate, stearic acid, titanium dioxide and talc.

The protease inhibitors that can be found in layer c) can be selected from: amino acids and their analogues, such as alpha-aminoboronic derivatives (boroleucine, boroalanine and borovaline), N-acetyl cysteine and pepstatylglutamic acid; peptides and derivatives, such as bacitracin, pepstatin, antipain, leupeptin, chymostatin, elastatinal, phosphoramidon, bestatin, puromycin and amastatin, ovomucoids, Bowman-Birk inhibitor (BBI), 1,10-phenanthroline; molecules with non-aminoacidic structures, such as organophosphorus inhibitors (di-isopropylfluorophosphate, phenylmethylsulphonylfluoride), 1,2,3,4-tetrahydro-1-naphtoate methanesulphonate (FK-448), camostat mesilate, gabexate mesilate, nafamostat mesilate, p-hydroxymercuribenzoate; bile salts, such as sodium glycocholate, sodium deoxycholate, sodium taurodeoxycholate; complexing agents, such as ethylenediaminetetraacetic acid (EDTA), diethylene triamine pentaacetic acid (DTPA), EGTA (ethylene glycol tetraacetic acid) and hydroxyquinoline. Absorption promoters that can, be contained in layer c) are chosen among: bile salts, such as sodium glycocholate, sodium glycodeoxycholate, sodium deoxycholate, sodium taurodeoxycholatc; dihydrofusidates, such, as sodium taurodihydro-fusidate, sodium glycodihydro-fusidate; surfactants, such as polyoxyethylene alkyl esters, polyoxyethylene alkyl eters, sodium lauryl sulfate, dioctyl sodium sulfosuccinate, N-lauryl-beta-D-maltopyranoside; complexing agents, such as EDTA, DTPA, EGTA; fatty acids, such as docosahexanoic acid, eicosapentaenoic acid, capric acid and salicylates. The binding material can be chosen among cellulose derivatives (for example hydroxypropyl methylcellulose, sodium carboxymethylcellulose), gums, starch, gelatine, polyvinylpyrrolidones and polyethylene glycols of various molecular weights.

The technique employed for the application of layers b) and d) of FIG. 1 can be carried out, for example, by film-coating, through which an aqueous solution of polymers or polymeric mixtures is applied onto the cores. The intermediate layer c), consisting of the protease inhibitor and/or absorption enhancer, can be applied onto the previously coated cores by film-coating applying an, aqueous solution containing the protease inhibitor and/or absorption enhancer and, optionally, the binding material or other pharmaceutically acceptable excipients.

Finally, the gastroresistant layer e) is generally composed of a polymer with pH-dependent solubility, such as a copolymer of methacrylic acid (for example Eudragit L, Eudragit FS; preferably Eudragit L30D55), or cellulose acetate phthalate (CAP), that can be applied by film-coating.

In an exemplary embodiment of the invention, illustrated in further detail in the following experimental section, the active ingredient is acetaminophen, the protease inhibitor is camostat mesilate, the inner and outer polymeric layers b) and d), respectively, are composed of hydroxypropyl methylcellulose and polyethylene glycol.

Aside from the above-mentioned methods, the formulations related to this invention can also be prepared with other methods available, such as double compression, powder layering or the ones described in Remington's: The Science and Practice of Pharmacy, 20^{th} Ed. Baltimore, Md., Lippincott Williams & Wilkins, 2000 and the ones described in Lieberman, H. A. and Lachman L. Editors, New York and Basil, Marcel Dekker, Inc., 1980.

### FIGURE DESCRIPTION

FIG. 1: Overview of the delivery system according to the invention: core a); internal layer b); intermediate layer containing the protease inhibitor and/or absorption enhancer c); external layer d); gastroresistant layer e).

FIGS. 2(a-c) 6(a-c): release profiles from systems containing acetaminophen and camostat mesilate with layers b) and d) of varying thicknesses (800 ml deionized water, 37 plus or minus 0.5 degrees Celsius, 31 cycles/minute, n=3).

### EXPERIMENTAL SECTION

### Materials and Methods

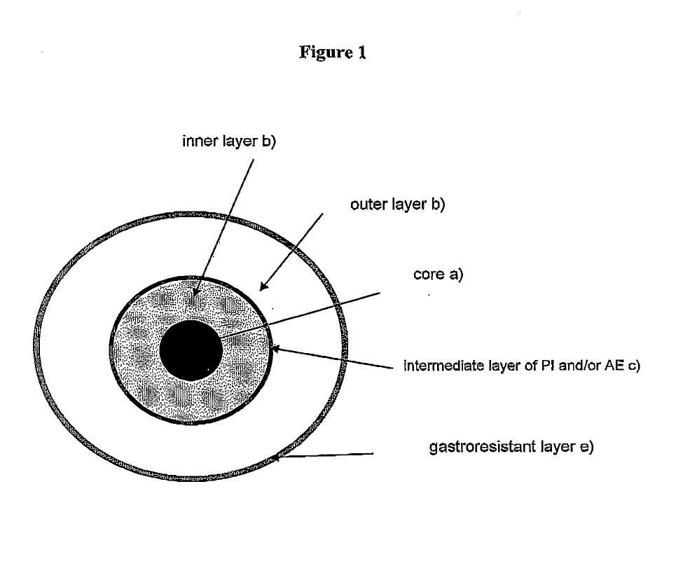

The hydrophilic layers b) and d) of FIG. 1 were obtained by top spray film-coating in fluid bed or rotating pan, applying an aqueous HPMC (8% w/w) and PEG 400 (0.8% w/w) solution onto the cores. The intermediate layer c), composed of camostat mesilate, was applied by film-coating in fluid bed, applying an aqueous solution of protease inhibitor and PEG 400 onto cores provided with the internal hydrophilic layer.

The process parameters employed for fluid bed film-coating are listed below: inlet air temperature 58-60 degrees Celsius; outlet air temperature 36-38 degrees Celsius, inlet air flap opening 40-45%; nozzle diameter 1.2 mm; nebulizing pressure 3.5-4.0 bar. During the coating process, the solution, kept at a constant temperature of 40 degrees Celsius in order to maintain an adequate viscosity, was carried to a two-way nebulizer by a peristaltic pump (VRX 88, Verder GmbH, D) with a neoprene tube (internal diameter=4.0 mm).

The process parameters employed for film-coating in rotating pan are listed below: ventilation air temperature 65 degrees Celsius; temperature of the cores 38-40 degrees Celsius; nebulizer pressure 0.4-0.6 bar; nozzle diameter 1.2 mm; distance between nebulizer and cores 8.5 cm; spray/non-spray ratio 30 sec-10 sec; spray rate 0.5 g/min; pan rotation speed 30 rpm.

The obtained systems were characterized in terms of weight, height and diameter. The thickness of the applied layers was calculated as follows:

thickness=(a/2+b/2)/2, where a is the change in diameter and b is the change in height.

The in vitro release profiles were evaluated by dissolution tests carried out in a disintegration test apparatus according to European Pharmacopoeia 0^{th} Ed. The dissolution medium was deionized water (800 ml) kept at 37 plus or minus 0.5 degrees Celsius. All release tests were carried out in triplicates.

Preparation of Systems Containing Acetaminophen and Camostat Mesilate

### Example 1

### Preparation of Systems Containing Acetaminophen and Camostat Mesilate (5 mg)—Thickness of the Inner Hydrophilic Layer b): 158 μm; Intermediate Layer c): 60 μm; Outer Hydrophilic Layer d): 546 μm

The core tablets were obtained by direct compression of powder mixtures containing acetaminophen (80% w/w), polyvinylpyrrolidone (2% w/w), microcrystalline cellulose (12.5% w/w), sodium carboxymethylstarch-Explotab (4.5% w/w), magnesium stearate (0.5% w/w) and colloidal silica (0.5% w/w).

The cores were coated in top spray fluid bed with an aqueous low-viscosity HPMC solution (Methocel E50, 8% w/w) containing PEG 400 (0.8% w/w) until a 13% weight gain (thickness 158 μm) was reached with respect to the starting substrate. These systems were further coated in rotating pan with an aqueous camostat mesilate solution (3.3% w/w) containing PEG 400 (0.3% w/w), in order to load 5 mg of camostat mesilate per tablet, and then subjected to the last coating process in top spray fluid bed with the same aqueous solution of hydrophilic polymer, until a 100% weight gain with respect to the starting tablets was obtained, approximately corresponding to an overall coating thickness of 800 μm.

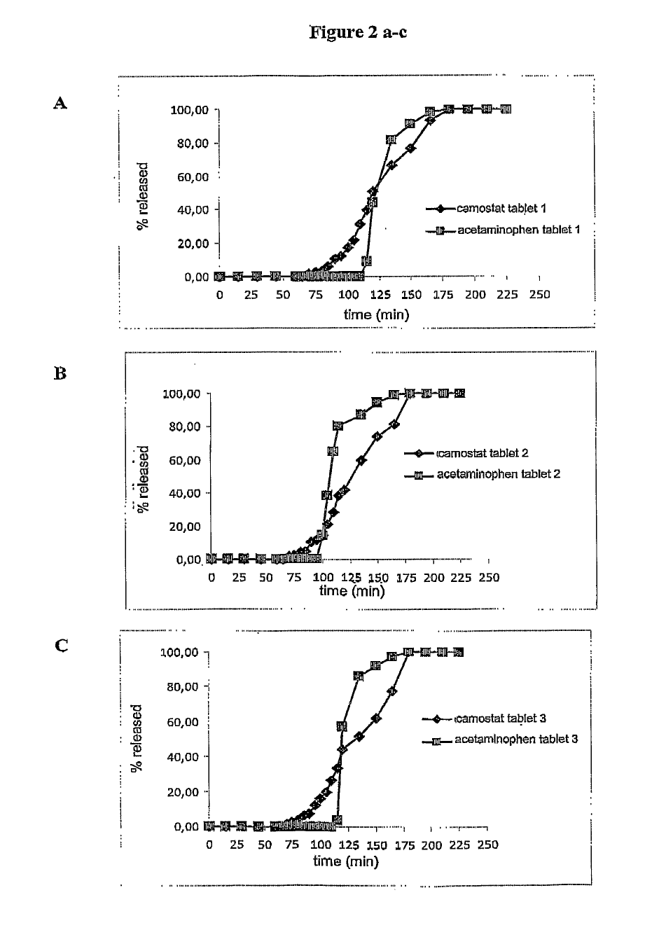

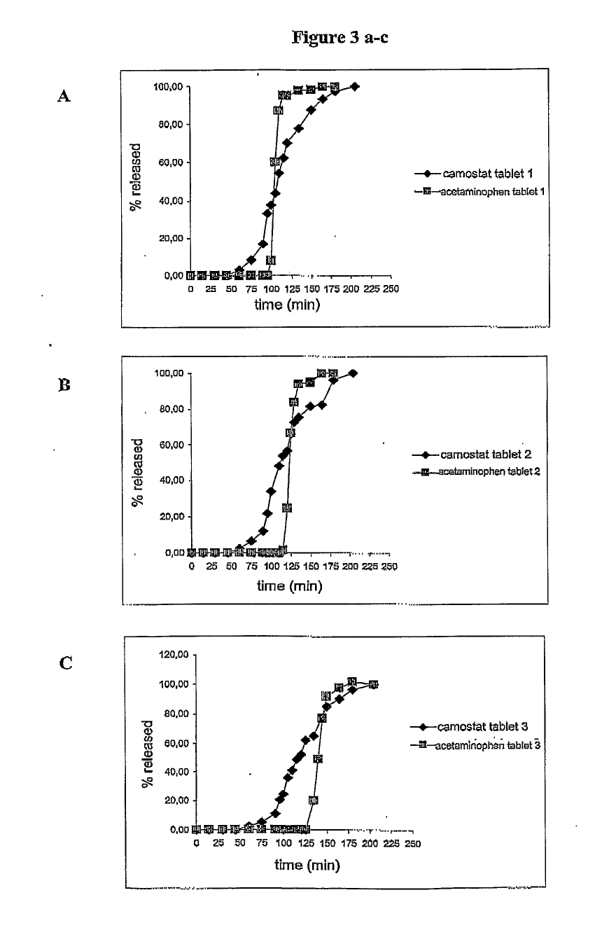

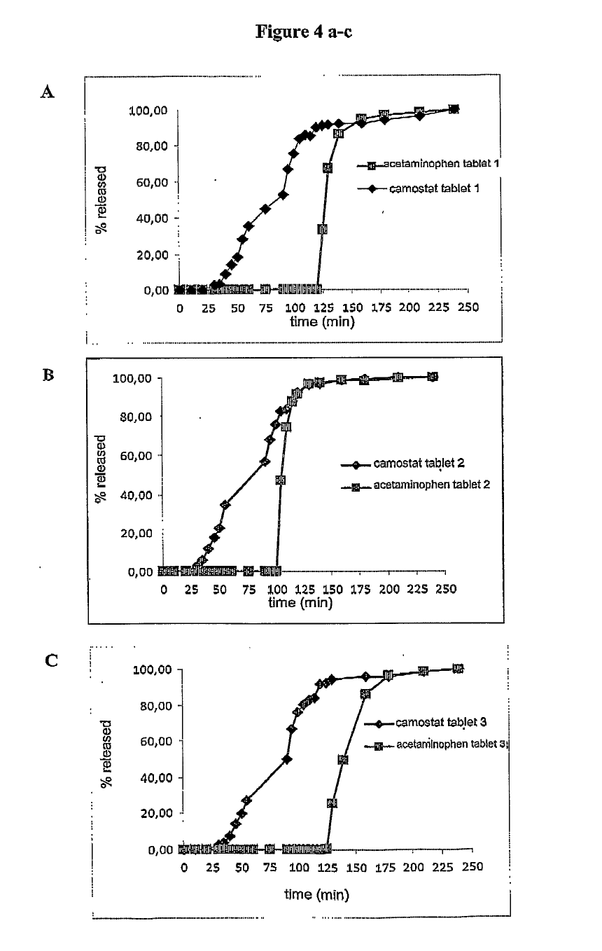

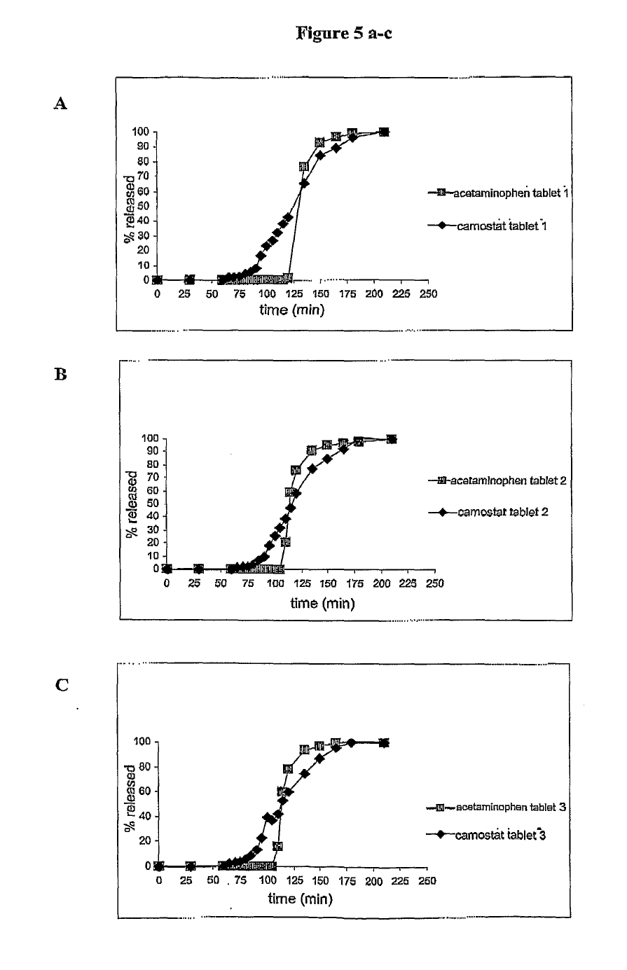

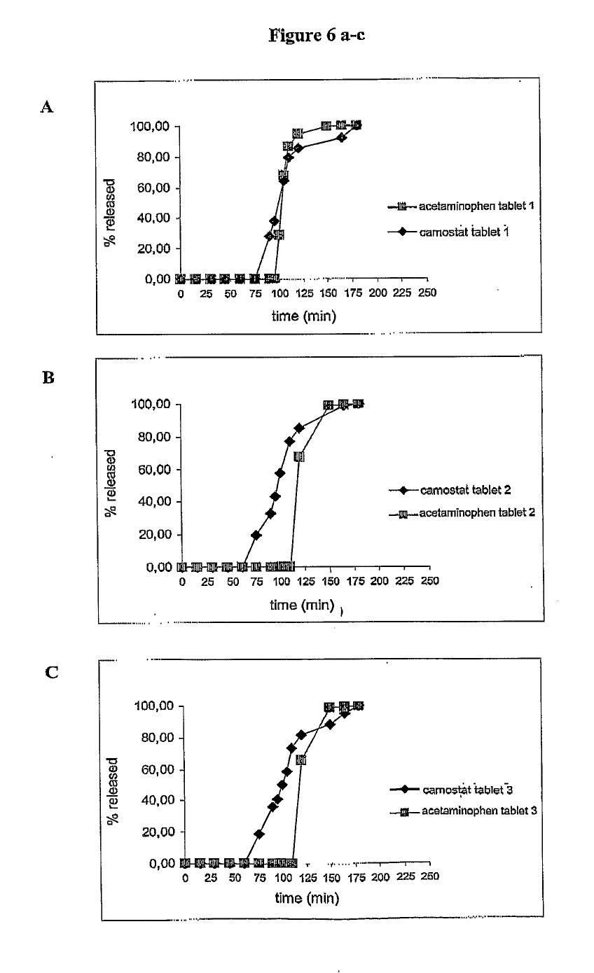

The release profiles are reported in FIG. 2 a-c.

Such systems gave rise to the release of camostat mesilate 20 minutes prior to that of acetaminophen. The temporal difference between the two release steps is attributed to the presence of the inner hydrophilic layer b) with a thickness of 158 μm.

### Example 2

### Preparation of Systems Containing Acetaminophen and Camostat Mesilate (5 mg)—Thickness of the Inner Hydrophilic Layer B): 248 μm, Intermediate Layer c): 83 μm; Outer Hydrophilic Layer d): 441 μm

The core tablets were obtained by direct compression of powder mixtures containing acetaminophen (80% w/w), polyvinylpyrrolidone (2% w/w), microcrystalline cellulose (12.5% w/w), sodiumcarboxymethylstarch-Explotab (4.5% w/w), magnesium stearate (0.5% w/w) and colloidal silica (0.5% w/w).

The cores were thus coated in top spray fluid bed with an aqueous low-viscosity HPMC solution (Methocel E50, 8% w/w) containing PEG 400 (0.8% w/w) until a weight gain of 25% (thickness 248 μm) with respect to the starting substrate was achieved. The resulting systems were subsequently coated in rotating pan with, an aqueous camostat mesilate (3.3% w/w) solution containing PEG 400 (0.3% w/w), in order to load 5 mg of camostat mesilate per tablet, and then subjected to the last coating process in top spray fluid bed with the same aqueous solution of hydrophilic polymer, until a 100% weight gain with respect to the starting tablets was obtained, approximately corresponding to an overall coating thickness of 800 μm.

The release profiles are reported in FIG. 3 a-c.

Such systems gave rise to the release of camostat mesilate 33 minutes prior to that of acetaminophen. The temporal difference between the two release steps is attributed to the presence of the inner hydrophilic layer b) with a thickness of 248 μm.

### Example 3

### Preparation of Systems Containing Acetaminophen and Camostat Mesilate (5 Mg)—Thickness of the Inner Hydrophilic Layer b): 434 μm; Intermediate Layer c): 66 μm; Outer Hydrophilic Layer d): 283 μm

The core tablets were obtained by direct compression of powder mixtures containing acetaminophen (80% w/w), polyvinylpyrrolidone (2% w/w), microcrystalline cellulose (12.5% w/w), sodium carboxymethylstarch-Explotab (4.5% w/w), magnesium stearate (0.5% w/w) and colloidal silica (0.5% w/w).

The cores were thus coated in top spray fluid bed with an aqueous low-viscosity HPMC solution (Methocel E50, 8% w/w) containing PEG 400 (0.8% w/w) until a weight gain of 50% (thickness 434 μm) with respect to the starting substrate was achieved. The resulting systems were subsequently coated in rotating pan with an aqueous camostat mesilate (3.3% w/w) solution containing PEG 400 (0.3% w/w), in order to load 5 mg of camostat mesilate per tablet, and then subjected to the last coating process in top spray fluid bed with the same aqueous solution of hydrophilic polymer, until a 100% weight gain with respect to the starting tablets was obtained, approximately corresponding to an overall coating thickness of 800 μm.

The release profiles are reported in FIG. 4 a-c.

Such systems gave rise to the release of camostat mesilate 75 minutes prior to that of acetaminophen. The temporal difference between the two release steps is attributed to the presence of the inner hydrophilic layer b) with a thickness of 434 μm.

### Example 4

### Preparation of Systems Containing Acetaminophen and Camostat Mesilate (10 Mg)—Thickness of the Inner Hydrophilic Layer b): 158 μm; Intermediate Layer c): 107 μm; Outer Hydrophilic Layer d): 527 μm.

The core tablets were obtained by direct compression of powder mixtures containing acetaminophen (80% w/w), polyvinylpyrrolidone (2% w/w), microcrystalline cellulose (12.5% w/w), sodium carboxymethylstarch-Explotab (4.5% w/w), magnesium stearate (0.5% w/w) and colloidal silica (0.5% w/w).

The cores were thus coated in top spray fluid bed with an aqueous low-viscosity HPMC solution (Methocel E50, 8% w/w) containing PEG 400 (0.8% w/w) until a weight gain of 13% (thickness 158 μm) with respect to starting substrate was achieved. The resulting systems were subsequently coated in rotating pan with an aqueous camostat mesilate (6.6% w/w) solution containing PEG 400 (0.7% w/w), in order to load 10 mg of camostat mesilate per tablet, and then subjected to the last coating process in top spray fluid bed with the same aqueous solution of hydrophilic polymer, until a 100% weight gain with respect to the starting tablets was obtained, approximately corresponding to an overall coating thickness of 800 μm.

The release profiles are reported in FIG. 5 a-c.

Such systems gave rise to the release of camostat mesilate 20 minutes prior to that of acetaminophen. The temporal difference between the two release steps is attributed to the presence of the inner hydrophilic layer b) with a thickness of 158 μm.

### Example 5

### Preparation of Systems Containing Acetaminophen and Camostat Mesilate (1 Mg)—Thickness of the Inner Hydrophilic Layer b): 248 μm; Intermediate Layer c): 20 μm; Outer Hydrophilic Layer d): 494 μm

The core tablets were obtained by direct compression of powder mixtures containing acetaminophen (80% w/w), polyvinylpyrrolidone (2% w/w), microcrystalline cellulose (12.5% w/w), sodium carboxymethylstarch-Explotab (4.5% w/w), magnesium stearate (0.5% w/w) and, colloidal silica (0.5% w/w).

The cores were thus coated in top spray fluid bed with an aqueous low viscosity aqueous HPMC solution (Methocel E50, 8% w/w) containing PEG 400 (0.8% w/w) until a weight gain of 25% (thickness 248 μm) with respect to the starting substrate was achieved. The resulting systems were subsequently coated in rotating pan with an aqueous camostat mesilate (3.3% w/w) solution containing PEG 400 (0.3% w/w), in order to load 1 mg of camostat mesilate per tablet, and then subjected to the last coating process in top spray fluid bed with the same aqueous solution of hydrophilic polymer, until a 100% weight gain with, respect to the starting tablets was obtained, approximately corresponding to an overall coating thickness of 800 μm.

The release profiles are reported in FIG. 6 a-c.

Such systems gave rise to the release of camostat mesilate 33 minutes prior to that of acetaminophen. The temporal difference between the two release steps is attributed to the presence of the inner hydrophilic layer b) with a thickness of 248 μm.

1. Solid pharmaceutical forms for oral administration comprising:
a) a core containing a drug susceptible to enzymatic degradation and/or poorly absorbed in the gastrointestinal tract;
b) an inner layer consisting of a polymer or a mixture of polymers that swell and/or dissolve and/or are degraded upon contact with the biological fluids in the gastrointestinal tract;
c) an intermediate layer consisting of a protease inhibitor and/or an absorption enhancer; and
d) an outer layer consisting of a polymer or a mixture of polymers that swell and/or dissolve and/or are degraded upon contact with the biological fluids in the gastrointestinal tract
wherein layer b) is from 10 to 1350 μm thick;
layer c) is from 10 to 1000 μm thick;
layer d) is from 150 to 1500 μm thick.

2. Pharmaceutical forms according to claim 1 wherein the inner layer b) is from 150 to 450 μm thick.

3. Pharmaceutical forms according to claim 1 wherein the intermediate layer c) is from 10 to 300 μm thick.

4. Pharmaceutical forms according to claim 1, wherein the outer layer d) is from 200 to 1000 μm thick.

5. Pharmaceutical forms according to claim 1, wherein the active ingredient is a peptide, a protein or an active ingredient that is susceptible to enzymatic degradation and/or poorly absorbed in the gastro-intestinal tract.

6. Pharmaceutical forms according to claim 5 wherein the protein is insulin.

7. Pharmaceutical forms according to claim 1, wherein the protease inhibitor is camostat mesilate.

8. Pharmaceutical forms according to claim 1, wherein the absorption enhancer is sodium glycocholate.

9. Pharmaceutical forms according to claim 1, wherein the inner layer is composed of a mixture of hydroxypropyl methylcellulose and polyethylene glycol 400.

10. Pharmaceutical forms according to claim 1, wherein said core is admixed with pharmaceutically acceptable excipients.

11. Pharmaceutical forms according to claim 1, wherein said intermediate layer is admixed with a binding polymer or other pharmaceutically acceptable excipient.

12. Pharmaceutical forms according to claim 1 further comprising an outer layer consisting of a gastro-resistant polymer.
